(19) United States
(12) Patent Application Publication
(10) Pub. No.: US 2024/0345049 A1
(43) Pub. Date: Oct. 17, 2024

(54) AQUATIC INSTALLATION FOUR-DIMENSIONAL MONITORING SYSTEM AND METHOD

(71) Applicant: PCFR SAS, Cucuron (FR)

(72) Inventors: Lane Hoy, Cucuron (FR); Xavier Darok, Cucuron (FR); Sebastien Ettling Coeffier, Cucuron (FR); Rene Brunier, Cucuron (FR); Lucas Dupuis, Cucuron (FR)

(21) Appl. No.: 18/446,087

(22) Filed: Aug. 8, 2023

Related U.S. Application Data
(63) Continuation-in-part of application No. 18/299,935, filed on Apr. 13, 2023.
(63) Continuation-in-part of application No. 18/300,030, filed on Apr. 13, 2023.
(63) Continuation-in-part of application No. 18/358,073, filed on Jul. 25, 2023.

(51) Int. Cl. G01N 31/22; G01N 21/78; G01N 33/18

(52) U.S. Cl. CPC G01N 31/221; G01N 21/78; G01N 33/18

(57) ABSTRACT

The aquatic installation four-dimensional monitoring system (100) comprises:
a submersible and/or floating vehicle (105, 106), comprising:
   a relative positioning coordinates acquisition means (110), configured to locate, in a three-dimensional space representative of the aquatic installation (111), the submersible vehicle and to provide the corresponding coordinates of the submersible and/or floating vehicle, and
   a water physical and/or chemical sensor (115), configured to provide a measure of a local physical and/or chemical state of the aquatic installation in the proximity of the submersible and/or floating vehicle, and
a timestamping means (120), configured to associate, with relative positioning coordinates acquired, a value representative of the time of acquisition, and
an aquatic installation local physical and/or chemical state information aggregation means (125), configured to associate timestamped coordinates to a local measured physical and/or chemical state.

### TECHNICAL FIELD OF THE INVENTION

The present invention relates to an aquatic installation four-dimensional monitoring system and an aquatic installation four-dimensional monitoring method. It applies, in particular, to the field of water treatment and to the field of in situ water treatment. The present invention applies to recreational aquatics (pools, spas, spray pads, water features, water fountains, water parks, wellness facilities, therapy facilities, lazy rivers, etc.) and to any similar industry or market segment (evaporative cooling for energy generation and data centers, heating, ventilation and air conditioning, and fire suppression stored water management, etc.).

### BACKGROUND OF THE INVENTION

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

In current systems, the efficiency of a water treatment circuit is measured within the hydraulic circuit (in the pipes, pumps and other chemical storing tanks) which supplies an aquatic installation.

However, such systems are inefficient in that they do not account for the shape of the installation, the circulation of water within that installation, the external parameters (such as weather forecasts), number and sizes of bathers (in the case of swimming pool), pollution in the aquatic installation (such as leaves, algae spots or stain) and the surrounding of the installation. Therefore, in order to monitor the impact of a water treatment process, operators must periodically test the physical and chemical state of the water in the aquatic installation in multiple different locations to determine whether the treatment process is successful or not and report the external factors and/or parameters. Such constraints also apply to the diagnostics to define a water treatment process to be performed and adjusted based on the external factors and/or parameters influence. Furthermore, such monitoring is also instantaneous and thus only provides a snapshot of the physical and/or chemical state of an aquatic installation. Furthermore, such monitoring is static in terms of positioning and unadaptable to increase the quality of a measure of a particular physical/chemical value correlated to the real time need of the aquatic installation taking into account any externals factors and/or parameters.

Therefore, current systems provide an inaccurate representation of the real-time and location dependent physical and/or chemical state and needs of water in an aquatic installation.

### SUMMARY OF THE INVENTION

The present invention aims at overcoming the above-mentioned drawbacks as well as other drawbacks that could be overcome although not mentioned in the description below.

The inventors have discovered that using an autonomous vehicle, configured to sense at least one parameter representative of the physical and/or chemical state of a body of water in proximity to the vehicle and associate such a value with a timestamp, both the local and global physical and/or chemical states of aquatic installation in an installation may be accurately monitored.

Such a monitoring system may be derived by adding various physical and/or chemical state computation means, based on the values sensed by the autonomous vehicle.

Such a monitoring system may be integrated in a feedback loop associating the autonomous vehicle and a hydraulic system associated with the aquatic installation.

Such a monitoring system may be associated with advanced computation capabilities, such as by using machine learning, to provide an accurate representation of the physical and/or chemical state of the water and/or diagnostics of water treatment processes to be performed on said body of water.

Such a monitoring system may ease facility management in a variety of contexts, such as swimming pools for example.

### BRIEF DESCRIPTION OF THE DRAWINGS

Other advantages, purposes and particular characteristics of the invention shall be apparent from the following non-exhaustive description of at least one particular system and method object of this invention, in relation to the drawings annexed hereto, in which:

FIG. 1 represents, schematically, a particular embodiment of a system object of the present invention,

FIG. 2 represents, schematically and in the form of a flowchart, a particular succession of steps of a method object of the present invention,

FIG. 3 represents, schematically, a particular embodiment of a submersible vehicle used in the system object of the present invention,

FIG. 4 represents, schematically, a particular embodiment of a floating vehicle used in the system object of the present invention,

FIG. 5 represents, schematically, a first view of a particular embodiment of a total alkalinity measurement device used in a system object of the present invention,

FIG. 6 represents, schematically, a second view of a particular embodiment of a total alkalinity measurement device used in a system object of the present invention,

FIG. 7 represents, schematically, a graph representing a succession of pH measurements at the boundary layer of a body of water for different total alkalinity values, and

FIG. 8 represents, schematically, a computing device capable of performing a method object of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted that the figures are not to scale.

In the present description, the terms “aquatic installation” refers to a body of water circulating in an installation and in which the two main sources of water removal are evaporation and active removal, via a hydraulic circuit, of the water. Such an aquatic installation refers to, for example, water in a swimming pool.

It should be noted that these terms, when used in relation to a physical and/or chemical parameter, may also refer to the aquatic installation itself or to an equipment or circuit associated with this installation.

Furthermore, while the present systems and devices are configured to be used in conjunction with aquatic installation, such aquatic installation is not restrictively itself a part of the present invention.

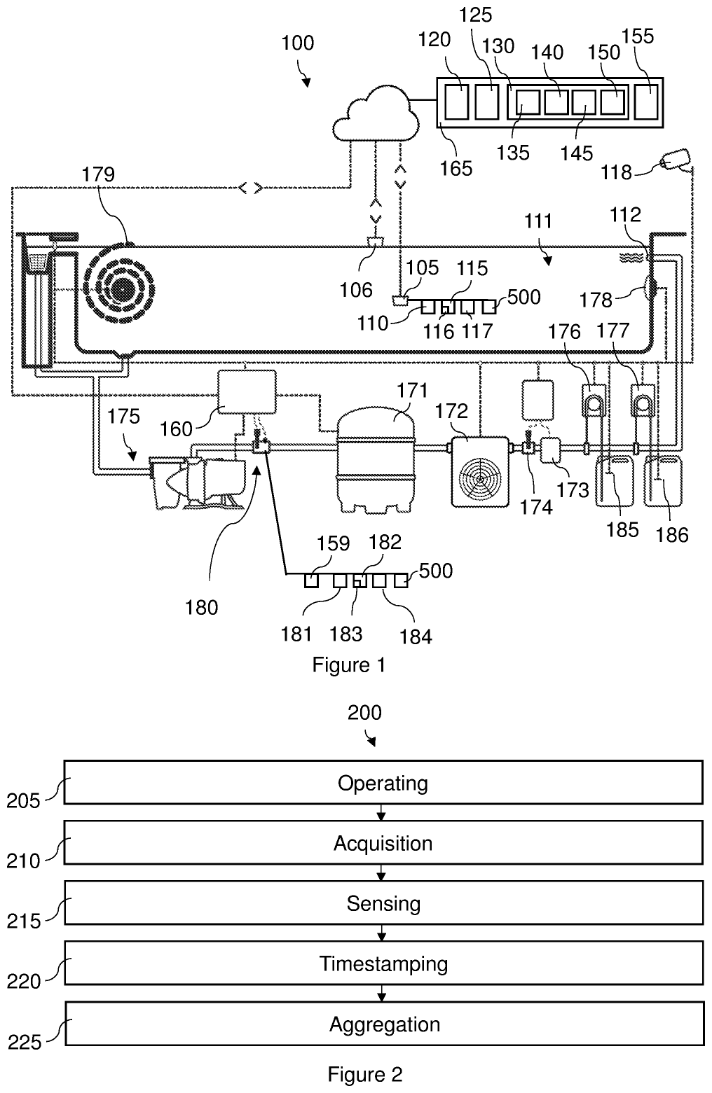

FIG. 1 represents, schematically, a particular embodiment of the system 100 object of the present invention. This aquatic installation four-dimensional monitoring system 100 comprises:
a submersible and/or floating vehicle, 105 and 106, comprising:
   a relative positioning coordinates acquisition means 110, configured to locate, in a three-dimensional space representative of the aquatic installation 111, the submersible vehicle and to provide the corresponding coordinates of the submersible and/or floating vehicle, and
   at least one physical and/or chemical sensor 115, configured to provide series of at least one sensed value representative of a local physical/chemical parameter in the proximity of the submersible and/or floating vehicle,
a timestamping means 120, configured to associate, with relative positioning coordinates acquired, a value representative of the time of acquisition, and
an aquatic installation physical and/or chemical state information aggregation means 125, configured to associate timestamped coordinates to a local measured physical and or chemical state.

In particular embodiments, the system 100 comprises at least one external sensor 118, configured to provide an external graphical representation and/or video and/or picture acquisition of water in the aquatic installation and/or the aquatic installation and/or to provide external factors and/or parameters acquisition.

The submersible and/or floating vehicle, 105 and/or 106, may correspond, for example, to any manually, remotely and/or automatically dirigible vehicle adapted to the particular use case.

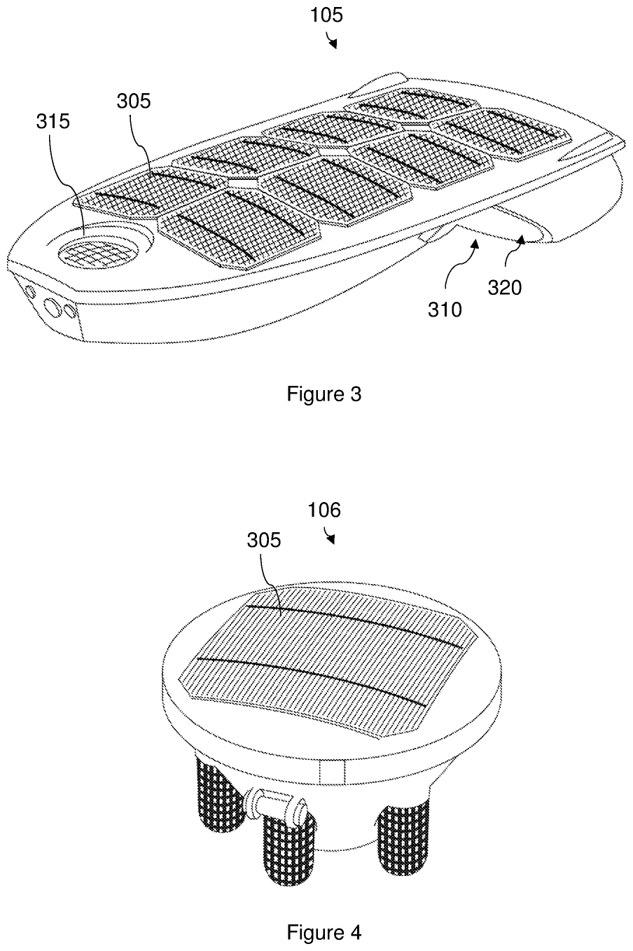

The vehicle 105 may correspond to a remotely or autonomously dirigible submarine vehicle, for example, such as shown in FIG. 3.

The vehicle 106 may also correspond to a floating pod, which is not dirigible, as shown in FIG. 4.

In particular embodiments, such as the one shown in FIG. 1, the system 100 comprises both a submersible vehicle (ROV) 105 and a floating vehicle 106.

In particular variants, at least one submersible and/or floating vehicle, 105 and/or 106, comprises a solar panel 305 configured to power an autonomous electricity source (not represented) and/or to charge the onboarded batteries (not represented).

In particular variants, at least one submersible and/or floating vehicle, 105 and/or 106, comprises an induction current collector configured to power an autonomous electricity source (not represented).

In particular variants, at least one submersible and/or floating vehicle, 105 and/or 106, comprises a power inlet configured to be connected to a charging wire to power an autonomous electricity source (not represented).

In particular variants, at least one submersible and/or floating vehicle, 105 and/or 106, comprises a propulsion 310 system, such as an engine associated with a boat propeller. Such a propulsion 310 system allows for the vehicle, 105 and/or 106, to move around in the water of the aquatic installation 111. This propulsion system 310 may comprise rear propellers 320, configured to generate forward, backward or yaw movements, and a front propeller 315, configured to generate upward or downward movements.

Such a submersible and/or floating vehicle, 105 and/or 106, further comprises a physical and/or chemical sensor 115. Such a physical and/or chemical sensor 115 is intended in the broadest sense, meaning that any physical and/or chemical parameter sensing device is encompassed, provided the output data of such a device is used to evaluate the physical and/or chemical state of the water in the basin 111, of basin 111 itself or of any other part of the aquatic installation.

The data provided by the physical/chemical sensor 115 may also be complemented by data originating from at least one further physical and/or chemical sensor, 181, 182, 183 and/or 184, is situated in an analysis chamber 180, or in a pipe of a circulation system where water flows, interacting with water in at least one aquatic installation and configured to provide series of at least one sensed value representative of a physical/chemical parameter. Such a physical/chemical parameter may be representative of a state of operation of the aquatic installation and/or of the water in the aquatic installation 111.

The physical and/or chemical sensor, 110, 115, 116, 117, 159, 181, 182, 183 and/or 184, is intended in the broadest sense, meaning that any physical and/or chemical parameter sensing device is encompassed, provided the output data of such a device is used to evaluate the physical and/or chemical state of the water in an aquatic installation 111 and/or the physical and/or chemical state of the aquatic installation 111, and/or status of equipment in the aquatic installation.

Such a water physical and/or chemical sensor, 110, 115, 116, 117, 159, 181, 182, 183 and/or 184, may correspond to, but is not limited to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

In particular embodiments, at least one water physical and/or chemical sensor 115 is an image acquisition means configured to acquire an image in which a color is representative of a local chemical state of the aquatic installation.

Such sensors may be used to provide an environmental context of data acquisition by the water physical and/or chemical sensor 115.

Such a submersible and/or floating vehicle, 105 and/or 106, further comprises a relative positioning coordinates acquisition means 110, configured to locate, in a three-dimensional space representative of the aquatic installation 111, the submersible vehicle, 105 and/or 106, and to provide the corresponding coordinates of the submersible and/or floating vehicle, 105 and/or 106.

Such a relative positioning coordinates acquisition means 110 is, for example, a sonar configured to provide distance values from edges of the aquatic installation 111. The distance values allow for the determination of the shape of the aquatic installation 111. Once the shape of the aquatic installation 111 is known, such distance values allow for the determination of the positioning of the submersible and/or floating vehicle, 105 and/or 106, within said installation 111.

In another variant, the relative positioning coordinates acquisition means 110 is, for example, a mechanical sensor used in coordination with a propulsion system 310 to map the shape of the aquatic installation 111 by detecting collisions of the submersible and/or floating vehicle, 105 and/or 106, with the edges of this installation 111.

Once the shape of the aquatic installation 111 is known, information originating from original parameters of the propulsion system 310 may be used to locate the submersible and/or floating vehicle, 105 and/or 106. For example, a duration of use of the propulsion system 310, associated with a power of propulsion, may be used in a calculation to determine a distance of the submersible and/or floating vehicle, 105 and/or 106, from the last known location.

The data resulting from the water physical and/or chemical sensor 115 and the relative positioning coordinates acquisition means 110 may be aggregated to form a timestamped water physical and/or chemical value sensed. This data may further be associated with environmental context values, such as water pressure or time of capture for example.

The timestamping means 120 may correspond, for example, to any electronic clock used by a computing device. Such a timestamping means 120 may be integrated into the submersible and/or floating vehicle, 105 and/or 106, or be remotely located from said submersible and/or floating vehicle, 105 and/or 106. By remotely located, it is intended that the timestamping means 120 is linked with the submersible and/or floating vehicle, 105 and/or 106, by a communication means, such as a peer-to-peer link or a communications network link, such as the Internet for example.

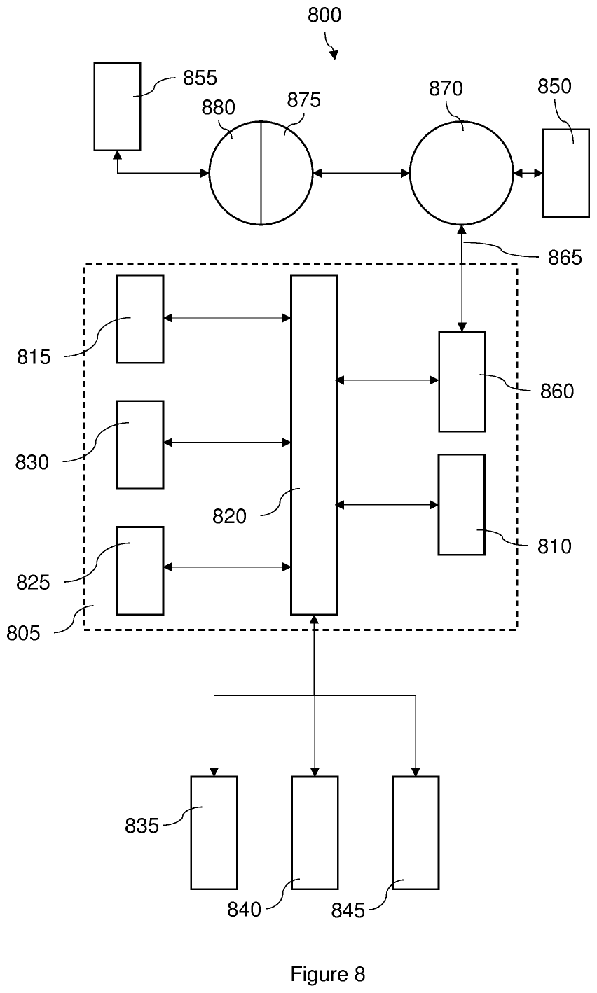

The aquatic installation local physical and/or chemical state information aggregation means 125 is, for example, a computer software executed upon a computing device such as illustrated in FIG. 8. This computing device is configured to associate, in a memory, the data resulting from the relative positioning coordinates acquisition means 110, the water physical and/or chemical sensor 115 and the timestamping means 120.

Such an association may be performed by concatenating said data in a single data stream or data frame or by creating a link between said data if such data is stored in separate database tables for example.

In particular variants, the submersible and/or floating vehicle, 105 and/or 106, comprises the aquatic installation local physical and/or chemical state information aggregation means 125.

In other variants, the aquatic installation local physical and/or chemical state information aggregation means 125 is remotely located from the submersible and/or floating vehicle, 105 and/or 106, and accessible via a communication means.

In particular embodiments, such as the one represented in FIG. 1, the system 100 object of the present invention comprises an aquatic installation physical parameter determination means 130, configured to determine a value representative of a parameter of the aquatic installation or of the water in said installation as a function of several aggregated aquatic installation local physical and/or chemical state information.

This aquatic installation parameter determination means 130 may be, for example, a computer software executed upon a computing device such as illustrated in FIG. 8. This aquatic installation parameter determination means 130 may use an algorithmic module or a machine learning module to link a parameter value to at least one value sensed by the submersible and/or floating vehicle, 105 and/or 106, and/or by any other sensor associated with the aquatic installation 111 and/or by any other sensor associated with a hydraulic circuit 175 associated with the aquatic installation 111.

An algorithmic module comprises a series of mathematical operations to be performed on a set or stream of data whereas a machine learning module comprises a machine learning architecture used on a training set or stream of data in order to produce a trained machine learning model that may then be used with operational data.

Such data represents, for example, physical and/or chemical sensed values, associated with time of measurement of said values.

In particular embodiments, such as the one shown in FIG. 1, the aquatic installation parameters determination means 130 comprises an aquatic installation physical and/or chemical state uniformity determination means 135, configured to determine a value representative of a physical and/or chemical state uniformity of the water in the aquatic installation 111 as a function of several aggregated aquatic installation local chemical state information.

In such embodiments, the aim is to have the same values in several locations in the installation. In the aquatic installation water there is always a different of values for any chemical parameters (such as pH, total alkalinity, disinfectant rate, etc.) linked to the water flow (area with a very low or almost no flow), the pollution content brought by users in the pool, and the products consumption. These differences generated concentration gradients which results in badly treated zones in the pool. Where total alkalinity is low (link to evaporation, or highly agitated water) the pH is less stable, and pH differences will occur. Where pH is higher than a set point (if we consider the set point to represent a pH at its water balance equilibrium value), the water step by step goes into a scaling state, which creates cloudy white water and scale deposition in the basin, pipes and equipment. In this case, chlorine will lose efficacy, and the area (mainly the surface) will be less disinfected.

On the contrary, when pH is lower than a set point (if we consider the set point to represent a pH at its water balance equilibrium value), the water step by step goes into an aggressive state, which damages the coating, and any equipment. In this case, the disinfectant has a higher efficacy, which could result in an extra oxidation level, creating attacks on coating and equipment.

In the case of high pollution brought by users, the disinfectant is consumed faster and generates disinfectant residues, which could become in excess if the disinfectant rate is low, or if its efficacy is not high enough. When in excess, these residues create irritations for users.

When the disinfectant rate is low, and its efficacy is low too, this helps the development of micro-organisms which could affect the health of users.

With the present system, it is possible to map the water in order to compensate any lack or excess of chemical rate to improve locally the water treatment. As an example, the lack of disinfectant could be compensated by adjusting the pump flow rate and/or injecting more disinfectant to help the good repartition of the disinfectant in the pool water and/or keep a perfect disinfection rate everywhere in the pool.

The term “physical and/or chemical state uniformity” refers to a measure of the variation in local physical and/or chemical state of the water in the aquatic installation 111. Such a variation may correspond to a gradient, for example. The less variation is detected, the higher the determined uniformity value is. For example, a physical and/or chemical state uniformity value of one may correspond to a ratio of maximum to minimum pH values of less than two for two different areas of the water in the aquatic installation 111.

The aquatic installation physical and/or chemical state uniformity determination means 135 may be, for example, a computer software executed upon a computing device such as illustrated in FIG. 8.

In particular embodiments, such as the one shown in FIG. 1, the aquatic installation parameter determination means 130 comprises a physical and/or chemical diffusion uniformity determination means 140, configured to determine a value representative of physical parameter and/or of density uniformity of a chemical compound in the aquatic installation 111 as a function of several aggregated aquatic installation local physical and/or chemical state information.

The term “diffusion uniformity” refers to a measure of the variation in local physical value and/or chemical composition of the water in the aquatic installation 111. Such a variation may correspond to a gradient, for example. This term may also refer to the local variation of concentration of a specific or a group of chemical compounds within the water of an installation 111. The less variation is detected, the higher the determined uniformity value is. For example, a uniformity value of one may correspond to a ratio of maximum to minimum concentration values of less than two for a specific chemical compound locally found in two different areas of the water in the aquatic installation 111, for example, or to a ratio of operating parameters (temperature, water clarity) of the installation.

The physical and/or chemical diffusion uniformity determination means 140 may be, for example, a computer software executed upon a computing device such as illustrated in FIG. 8.

In particular embodiments, such as the one shown in FIG. 1, the aquatic installation parameter determination means 130 comprises a physical value and/or chemical compound flow determination means 145, configured to determine a value representative of the flow of a physical and/or a chemical compound in the aquatic installation 111 as a function of several aggregated aquatic installation local physical and/or chemical state information.

The term “flow” refers to a measure of a water physical parameter or chemicals in the aquatic installation 111. Such a value for the flow may be obtained as a function of water pressure operated on the submersible and/or floating vehicle, 105 and/or 106. Such a value for the flow may be obtained as a function of the evolution, over time, in concentration of a specific physical parameter (such as temperature or turbidity) and/or chemical compound, group of chemical compounds, or chemical state (such as pH) in a set of locations in the aquatic installation 111. Such a value may be performed based on a series of such measurements in several locations and for a series of chemicals added to the water of the aquatic installation 111.

The physical parameter and/or chemical compound flow determination means 145 may be, for example, a computer software executed upon a computing device such as illustrated in FIG. 8.

In particular embodiments, such as the one shown in FIG. 1, the aquatic installation physical parameter determination means 130 comprises a risk zone determination means 150, configured to determine a value representative of a risk relative to the local chemical state in at least part of the aquatic installation as a function of several aggregated aquatic installation local physical and/or chemical state information.

Such a risk zone may correspond to a difference for a measured parameter from a target value that exceeds a determined threshold value, such as, for example:
pH different from sent point (offset included), and/or
disinfectant rate different from sent point (offset included), and/or
total alkalinity different from sent point (offset included).

The risk zone determination means 150 may be, for example, a computer software executed upon a computing device such as illustrated in FIG. 8.

In particular embodiments, a computing device, such as a remote computing device 165, is configured to build and render a virtual representation of the aquatic installation 111 and the aggregated data. This allows for providing a computer software which allows users to monitor the evaluation of the physical and/or chemical state of the water in the aquatic installation 111 at different times. The use of specific color codes allows for providing advanced analytics, such as showing the presence of several physical parameters and/or chemical compounds in one representation of the water of the aquatic installation 111, where each color is associated to a physical parameter value and/or to the presence and/or concentration of a distinct chemical compound.

In particular embodiments, such as the one shown in FIG. 1, the system 100 object of the present invention comprises a command emitter 155 configured to emit a command representative of a target operational value for an actuator 160 interacting with the physical and/or chemical state of the aquatic installation.

The command emitter 155 is, for example, a computer software executed upon a computing device, such as illustrated in FIG. 8, and associated to a communication means linking the emitter 155 to the actuator 160.

This command emitter 155 is, for example, activated as a function of the result of a comparison between the value of a parameter sensed by the submersible and/or floating vehicle, 105 and/or 106, and a predetermined target value. Such a target value corresponds to, for example, a value representative of a desired physical and/or chemical state of the water in the aquatic installation 111.

Such a command may correspond to, for example:
a release or the end of a release of a chemical compound in the water of the aquatic installation 111, and/or
an increase or a decrease in a water pump activation, and/or
an increase or a decrease in water heater activation.

As it can be understood, in particular embodiments, such as the one shown in FIG. 1, the system 100 object of the present invention may comprise a remote computing device 165, comprising at least one of:
the timestamping means 120, and/or
the information aggregation means 125, and/or
the physical and/or chemical parameter determination means 130.

The remote computing device 165 is, for example, accessible in the cloud via communication means.

The remote computing device 165 can be configured to control, register and adjust a set of predetermined installation 111 operational parameters values (such as the pH or alkalinity for example).

The remote computing device 165 may be, for example, operated as such:
all the data is routed by a traffic manager to a dedicated cloud (computing device and/or computer software):
   in each computing device and/or computing software, an algorithm manages the data's individual value from the camera acquisition of at least one vehicle, 105 and/or 106, and
   in a second step, an algorithm manages a combined global data value, and sends this data to a big data management module using the traffic manager, and
the global data from at least one vehicle, 105 and/or 106, and an external camera are compared to a user-set point:
   in case of differences between these values, the aquatic installation 111 operational parameters are adjusted using water treatment and equipment piloting improvements, and
   in parallel, a timestamped three-dimensional mapping of the aquatic installation 111 is recorded, and risk-determining algorithms are executed to determine the presence and severity of risks in the water installation 111 a plurality of zones, and
   a timestamped three-dimensional map is generated comprising a color code for the pool risks to identify the risk prone zones in a simplified manner.

As it can be understood, in particular embodiments, such as the one shown in FIG. 1, the system 100 object of the present invention may comprise at least a sensor 159 and/or actuator 160 configured to interact with an element 180 of an installation sanitation circuit 175 associated to the aquatic installation 111, said sensor and/or actuator being activated as a function of at least one aggregated aquatic installation local physical and/or chemical state information.

Specifically, the element referenced 180 refers to a chamber in which a sample of the water traversing the hydraulic circuit 175 is analyzed by a sensor 159. Such a sensor 159 may correspond to an ORP sensor or alkalinity sensor. Such values may be compared with the values sensed by the submersible and/or floating vehicle, 105 and/or 106, and optionally adjusted with a determined offset value corresponding to a standard difference between a point of treatment and a point within the installation.

Another element may correspond to a filter 171 analyzed by a sensor (not represented). Such a sensor may be adapted to provide values representative of the water pressure or a level of dirt accumulated in the filter 171. This filter 171 may further be associated to an actuator (160). Such an actuator may be configured to automatically clean the filter. Such an actuator may be linked to a remote computing device 165.

Another element may correspond to a heat pump 172 associated to an actuator (160) configured to increase or decrease the temperature of the water. Such an actuator may be linked to a remote computing device 165.

Another element may correspond to a disinfectant device 173 (electro-chlorinator system, ultraviolet system, or ozone generator system or any possible in situ disinfectant generator) associated to an actuator (160) configured to increase or decrease the activation of the disinfectant device 173. Such an actuator may be linked to a remote computing device 165.

Such a disinfectant device 173 may further be associated with an analysis chamber 174 associated with a sensor (not referenced). Such a sensor may be configured to detect a level of bacterial activity in a sample of water from the aquatic installation 111.

Another element may correspond to a pH adjustment device 176 associated to an actuator (160) configured to increase or decrease the pH of the water. Such an actuator may be linked to a remote computing device 165.

Such a pH adjustment device 176 may be associated to a pH sensor (not represented) or a pH adjustment chemical compound drum level 185.

Another element may correspond to a disinfectant (such as liquid chlorine or sodium hypochlorite or any other liquid disinfectant) release device 177 associated to an actuator (160) configured to increase or decrease the concentration of disinfectant in the water. Such an actuator may be linked to a remote computing device 165.

Such a disinfectant release device 177 may be associated to a disinfectant chemical compound drum level 186.

Another element may correspond to a light 178 configured to illuminate the water in the basin 111 and associated to an actuator (160) configured to activate or deactivate the light 178. Such an actuator may be linked to a remote computing device 165. The activation of such a light 178 increases the performance of image-based sensors, such as particle sensors.

Another element may correspond to an installation cover 179 configured to selectively cover the aquatic installation 111.

This installation cover 179 may be associated with an actuator (160) configured to open or close the cover 179, for purposes of safety, evaporation control, and energy cost reduction for example. Such an actuator may be linked to a remote computing device 165.

This installation cover 179 may be associated with a sensor (not referenced) configured to monitor the position of the cover 179.

In particular embodiments, such as the one shown in FIG. 1, the system 100 object of the present invention further comprises an external image capture device 118, such as a video camera for example. This image capture device is configured to monitor the aquatic installation 111 so as to provide additional data in relation to the water in the aquatic installation 111 or the status of the system 100, and notably the submersible (ROV) and/or floating vehicle, 105 and/or 106, within the aquatic installation 111. Such data may correspond to infrared image capture, the detection of a number of bathers in the aquatic installation 111, the presence of pollution in the aquatic installation 111, a coating aspect of the aquatic installation 111, a water clarity of the aquatic installation 111 and position of the submersible and/or floating vehicle, 105 and/or 106.

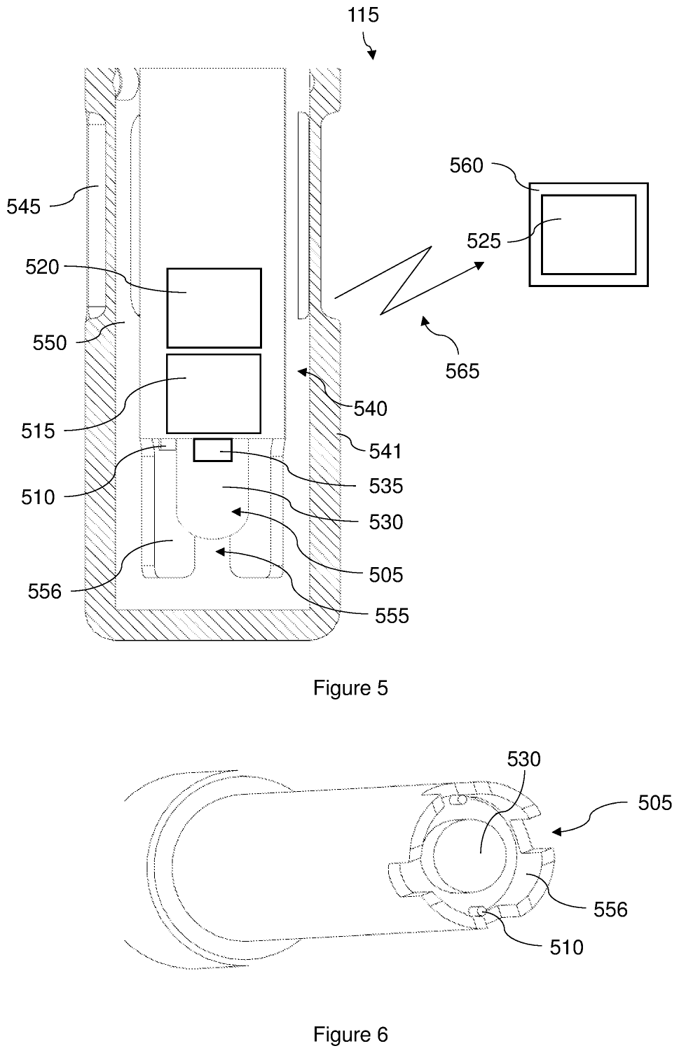

In particular embodiments, the system 100 comprises a total alkalinity measurement device, such as shown in FIGS. 5 and 6, comprising:
a pH probe 505 configured to measure pH at the boundary layer of a body of water, corresponding to the water physical and/or chemical sensor 110 of FIG. 1,
a floating reference device 510 in proximity of the pH probe,
a probe controller 515, configured to sequentially activate and deactivate the pH probe,
a pH measurement variation detection device 520, configured to detect a variation of pH measurement in a sequence of pH probe measurements, and
an aquatic total alkalinity value determination device 525, configured to determine an aquatic total alkalinity value of the body of water as a function of the pH measurement variation detected.

The pH probe 505 can be of any type known to a person skilled in the art that is suited for the particular implementation and intended use of the system 500. Such a pH probe 505 may differ in nature depending on the context of use of the system 500. For example, in a swimming pool, the pH probe 505 may comprise an oxidation-reduction potential sensor 535.

The objective of the pH probe 505 is to allow for the reproductible measurement of the pH in a body of water. Such a pH probe 505 is typically electronic and requires the supply of electrical energy to function. Such a pH probe 505 may further comprise a digital switch, allowing for the selective activation/deactivation of at least part of the core components of the pH probe 505.

The pH probe 505 may be mechanically located at the distal end of a sensor body, such as shown in FIGS. 5 and 6. The purpose of such a sensor body is the insertion in the body of water and, in preferred embodiments, within an analysis chamber 540.

The reference floating device 510, sometimes called “solution earth” or “liquid junction”, can correspond to any electrically conductive electrode or pin configured to normalize the signal sensed by the pH probe 505, avoiding electric noise in the proximity of the pH probe 505.

In the example shown in FIGS. 5 and 6, the reference floating device 510 comprises two electrodes, each located on a different side of a sensor 535 of the probe 505. Such electrodes may be diametrically opposed, with the sensor 535 acting as the center of a circle in which both electrodes are located on the periphery of said circle, for example. Such electrodes and the sensor 535 may be geometrically aligned.

In particular embodiments, such as the one shown in FIG. 5, the pH probe 505 comprises:
the floating reference device 510,
a microporous glass bulb membrane 530, and
an oxidation-reduction potential sensor 535.

pH measurement is based on the relationship between the concentration of H+ ions in tested water and the difference in electrochemical potential which is established in the lead-free glass bulb membrane of the probe. This lead-free bulb is specifically designed to be selective to H+ ions concentration.

In general, the pH probe 505 is made of a simple electronic amplifier and a combined electrode, which consists of two electrodes: one whose potential is known and constant and the other whose potential varies with the pH.

Once the probe 505 is in contact with water, the H+ ions exchange on the glass bulb, creating an electrochemical potential across the bulb. The electronic amplifier detects the difference in electrical potential between the two electrodes generated in the measurement and converts the potential difference to pH units.

The pH value is determined by correlation because the potential difference between the two electrodes evolves proportionally to the pH according to the Nernst equation.

The probe controller 515 is, for example, an electronic circuit configured to electrically or electronically activate and deactivate, or connect and disconnect, the pH probe 505 or the sensor of said pH probe 505. Such an activation/deactivation or connection/disconnection may be performed by cutting and restoring power supply to the pH probe 505 or sensor or by emitting an activation/deactivation or connection/disconnection command to said pH probe 505 or sensor or relay.

The terms “activate and deactivate” relate to any hardware or software level activation/deactivation and/or to the connection/disconnection of the pH probe 505.

The probe controller 515 may itself be activated as a function of a command emitted by a computing device, located on site and mechanically connected to the pH probe 505 and/or the probe controller 515 or remotely located and connected to the probe controller 515 by way of a data connection.

The probe controller 515 may comprise, for example, a computer software executed upon a computing device, said computer software triggering the activation/deactivation or connection/disconnection of the pH probe 505. Such a computer software may correspond, for example, to a particular firmware or driver. Such a computer software may be updated remotely, and such an update may be automatically installed in the system 500.

The probe controller 515 may be configured to activate or connect the pH probe 505 periodically. The pH probe may be activated or physically connected, for example, every 60 seconds. Such an activation or physical connection may be conditional, for example to the activation of a water displacement pump. The rate of measurement may be variable depending on the mode configured. The duration of measurement may depend on the water's stability, so the pH measurement last until the measured pH is sufficiently stable.

Such an activation/deactivation can be performed by an electronic relay.

The pH measurement variation detection device 520 is, for example, an electronic device associated with the pH probe 505, configured to record a succession of pH values measured by the pH probe 505 and to compute, from said succession, a measurement variation value. Such a measurement variation value may be computed by the subtraction of a recent value from an older value.

The measured variation may be performed on immediately subsequent measured pH values or be sampled according to a particular sampling rule. Such variation may also be performed on an aggregate values of measured pH values.

For example, the pH measurement variation detection device 520 may be configured to subtract the average measured pH value during a specific, more recent timeframe from the average measured pH value during a specific, older timeframe.

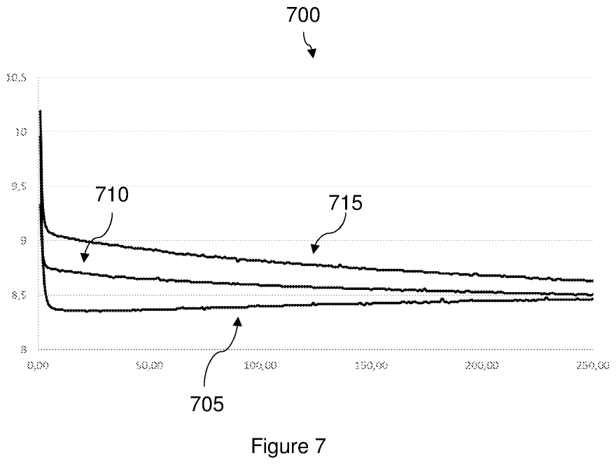

For example, the pH measurement variation detection device 520 may be configured to compute determine a mathematical function fitting a succession of data points linking measured pH to time of measurement since an initial measurement. Such an example is shown in FIG. 7. In other examples, the pH measurement variation detection device 520 may be configured to store, in a memory, a succession of data points linking measured pH to time of measurement since an initial measurement.

The system 500 may further comprise a timestamping means, configured to associate a time of measurement to a sensed pH value by the pH probe 505.

The action of repeatedly measuring the pH in the same water sample induces variations in the measurement of pH, the magnitude of these variations being dependent on the total alkalinity of the water. Such a pH measurement variation detection device 520 may also correspond to a computer software executed upon a computing device.

The pH measurement variation detection device 520 may operate remotely from the pH probe 505. In such a case, the system 500 may further comprise a communication means 565 to transmit data from the pH probe 505 to the pH measurement variation detection device 520. In such a case, the pH measurement variation detection device 520 may correspond to a computer program executed by a computing server, accessible on the cloud, via a data network such as the Internet for example.

The aquatic total alkalinity value determination device 525 is, for example, an electronic device associated with the pH measurement variation detection device 520, configured to associate a total alkalinity value to the measured variation.

For example, the total alkalinity value determination device 525 may be configured to compute the derivative of a mathematical function fitting a succession of data points linking measured pH to time of measurement since an initial measurement. Such an example is shown in FIG. 7.

The total alkalinity value determination device 525 may be configured to associate, with specific or ranges of said derivatives, a specific or a range of total alkalinity value.

For example, in FIG. 7:
a first series 705 of pH measurements (Y-axis), at specific times (X-axis), measured in minutes, since an initial measurement, for a total alkalinity value of 220 mg/l,
a second series 710 of pH measurements (Y-axis), at specific times (X-axis), measured in minutes, since an initial measurement, for a total alkalinity value of 125 mg/l, and
a third series 715 of pH measurements (Y-axis), at specific times (X-axis), measured in minutes, since an initial measurement, for a total alkalinity value of 19 mg/l.

Obtaining such series linking pH to alkalinity value relationship can be performed by empirically measuring, for different values of total alkalinity and a determined activation/connection frequency for the pH sensor, values of pH in the boundary layer of a body of water and storing these series in a memory. The number of such tests to be performed is limited in terms of scope, considering the limited number of values for alkalinity.

Such a total alkalinity value may be a mathematical function of the measured variation. Such a mathematical function may be performed by determining a regression function based upon the pH series captured, or derivative values of these series, as well as the operational parameters associated with the capture.

Such derivative values may be, for example, any type of averages or parameters of derivative functions.

For example, the following mathematical formula may be used:

$$A l k = - 0.0001 ( A V {G}_{1} - A V {G}_{2} ) + 0.1468$$

Where:
Alk designates the total alkalinity value,
AVG₁ designates the average pH values measured from 20 seconds to 80 seconds after the initial measurement,
AVG₂ designates the average pH values measured from 300 seconds to 360 seconds after the initial measurement, and

Such a function may be approximated to Alk=(AVG₁−AVG₂).

From such a function, the following correspondence table may be obtained:

| Total alkalinity value | AVG₁ − AVG₂ |
|---|---|
| 10 | 0.1368 |
| 20 | 0.1268 |
| 30 | 0.1168 |
| 40 | 0.1068 |
| 50 | 0.0968 |
| 60 | 0.0868 |
| 70 | 0.0768 |
| 80 | 0.0668 |
| 90 | 0.0568 |
| 100 | 0.0468 |
| 110 | 0.0368 |
| 120 | 0.0268 |
| 130 | 0.0168 |
| 140 | 0.0068 |
| 150 | −0.0032 |
| 160 | −0.0132 |
| 170 | −0.0232 |
| 180 | −0.0332 |
| 190 | −0.0432 |
| 200 | −0.0532 |

Such a total alkalinity value may be determined as a function of the measured variation and a preset threshold value, representative of a particular total alkalinity value.

Such an aquatic total alkalinity value determination device 525 may also correspond to a computer software executed upon a computing device.

The aquatic total alkalinity value determination device 525 may operate remotely from the pH probe 505 and/or the pH measurement variation detection device 520. In such a case, the system 500 may further comprise a communication means 565 to transmit data from the pH measurement variation detection device 520 to the aquatic total alkalinity value determination device 525. In such a case, the aquatic total alkalinity value determination device 525 may correspond to a computer program executed by a computing server, accessible on the cloud, via a data network such as the Internet for example.

In particular embodiments, the pH probe controller 515 is configured to sequentially activate and deactivate, or connect and disconnect, the pH probe 505 in a body of water with no flow. Such a state may be reached by stopping a pumping system introducing water in the body of water. In particular variants, the pH probe 505 may be activated after an absence of flow is detected (by a flow sensor, for example). In particular variants, a chamber in which the pH probe 505 is located may comprise valves that may be closed prior to the operation of the pH probe 505 activation/deactivation or connection/disconnection sequence.

The terms “body of water with no flow” designate a body of water with limited water flowing. In such a body of water, the water may circulate, but limited new water may enter.

In particular embodiments, the pH probe 505 is configured to be positioned in a small-volume body of water. Such a small volume may correspond to, for example, 1 to 2 milliliters.

The terms “small-volume body of water” designate a body of water in which the chemical reaction taking place during an interval of deactivation/activation, or connection/disconnection, of the pH probe 505 provides significant impact on the pH measure so as to show a variation between two successive measurements of the pH by the pH probe 505.

In particular embodiments, the system 500 object of the present invention comprises an analysis chamber 540, comprising an opening 545, a main volume 550 connected to the opening 545 and a recess 555 in the main volume 550, the pH probe 505 being in contact with the water in the recess 555.

The analysis chamber 540 may comprise a sensor housing 541 delimiting an internal volume in which the pH probe 505 or a sensor body associated with said pH probe 505 may be inserted.

The analysis chamber 540 is preferably configured to limit the flow of water and the volume of water in proximity to the pH probe 505. Such a configuration may be performed by selecting dimensions that limit the quantity of water entering the analysis chamber 540.

The analysis chamber 540 comprises an opening 545, of arbitrary dimensions, which allows for the passage of water from the body of water to the proximity of the pH probe 505.

The analysis chamber 540 comprises a main volume 550, defined for example by the interior dimensions of the sensor housing 541.

The analysis chamber 540 comprises a recess 555, defined by a subset of the interior dimensions of the sensor housing 541. In particular embodiments, the recess 555 is formed by crenellated sensor body extensions 556 associated to the pH probe 505, said crenellated sensor body extensions 556 limiting the movement of water in the proximity of the pH probe 505.

There are many possible configurations of the analysis chamber 540. Such configurations preferably limit the quantity of water in proximity to the pH probe 505 and/or limit the movement of water in proximity of the pH probe 505.

In particular embodiments, the system 500 object of the present invention comprises a remote computing device 560 comprising the aquatic total alkalinity value determination device 525 and a communication means 565 between the pH measurement variation detection device 520 and the aquatic total alkalinity value determination device 525.

Such a remote computing device 560 may correspond to, for example, a computing server hosted remotely and accessible through a data network, such as the Internet for example.

In particular embodiments, the aquatic total alkalinity value determination device 525 operates an algorithm and/or a trained machine learning model to associate an aquatic total alkalinity value with a variation in measured pH.

In particular embodiments, the pH probe 505 is configured to measure the pH of the body of water in a swimming pool.

In particular embodiments, the pH probe 505 is configured to measure the pH of the body of water in a pipe.

FIG. 2 represents, schematically, a particular succession of steps of the method 200 object of the present invention. This aquatic installation four-dimensional monitoring method 200 comprises:
a step 205 of operating a submersible and/or floating vehicle to navigate in an aquatic installation,
a step 210 of relative positioning coordinates acquisition to locate, in a three-dimensional space representative of the aquatic installation, the submersible vehicle and to provide the corresponding coordinates of the submersible and/or floating vehicle,
a step 215 of physical and/or chemical sensing to provide series of at least one sensed value representative of a local physical/chemical parameter in the proximity of the submersible and/or floating vehicle,
a step 220 of timestamping to associate, with relative positioning coordinates acquired, a value representative of the time of acquisition and
a step 225 of aquatic installation local physical and/or chemical state information aggregation to associate timestamped coordinates to a local measured physical and/or chemical state.

Particular embodiments of these steps are disclosed in regard to FIG. 1.

As it can be understood:

FIG. 1 further represents a particular embodiment of the system object of the present invention, which comprises a total alkalinity regulation unit, configured to increase or decrease the total alkalinity of the body of water as a function of the measured total alkalinity value and a target total alkalinity value.

The total alkalinity regulation unit may correspond to any device suited for the regulation of alkalinity known to one skilled in the art. Such a device may be, for example, a mixer configured to mix acid from a reservoir and a stream of water processed in a swimming pool water treatment circuit.

The total alkalinity regulation unit operates to reach a target total alkalinity in the body of water. This target total alkalinity can be set by a user or be remotely set by a computing system. Such a value may be higher or equal to 120 mg/l (or ppm) and lower than 250 mg/l (or ppm). If the measured total alkalinity is above the target total alkalinity, the regulation unit may be operated to reduce the total alkalinity in the body of water whereas if the measured total alkalinity is below the target total alkalinity, the regulation unit may be operated to increase the total alkalinity in the body of water.

FIG. 8 represents a block diagram that illustrates an example computing device, 800, such as the computing device 165 disclosed in regard of FIG. 1, with which an embodiment of the present invention may be implemented. In the example of FIG. 8, a computer system 805 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 805 includes an input/output (IO) subsystem 820 which may include a bus and/or other communication mechanism(s) for communicating information and/or instructions between the components of the computer system 805 over electronic signal paths. The I/O subsystem 820 may include an I/O controller, a memory controller and at least one I/O port. The electronic signal paths are represented schematically in the drawings, for example as lines, unidirectional arrows, or bidirectional arrows.

The computer system 805 includes an input/output (IO) subsystem 820 which may include a bus and/or other communication mechanism(s) for communicating information and/or instructions between the components of the computer system 805 over electronic signal paths. The I/O subsystem 820 may include an I/O controller, a memory controller and at least one I/O port. The electronic signal paths are represented schematically in the drawings, for example as lines, unidirectional arrows, or bidirectional arrows.

At least one hardware processor 810 is coupled to the I/O subsystem 820 for processing information and instructions. Hardware processor 810 may include, for example, a general-purpose microprocessor or microcontroller and/or a special-purpose microprocessor such as an embedded system or a graphics processing unit (GPU) or a digital signal processor or ARM processor. Processor 810 may comprise an integrated arithmetic logic unit (ALU) or may be coupled to a separate ALU.

Computer system 805 includes one or more units of memory 825, such as a main memory, which is coupled to I/O subsystem 820 for electronically digitally storing data and instructions to be executed by processor 810. Memory 825 may include volatile memory such as various forms of random-access memory (RAM) or other dynamic storage devices. Memory 825 also may be used for storing temporary variables or other intermediate information during execution of instructions to be executed by processor 810. Such instructions, when stored in non-transitory computer-readable storage media accessible to processor 810, can render computer system 805 into a special-purpose machine that is customized to perform the operations specified in the instructions.

Computer system 805 further includes non-volatile memory such as read only memory (ROM) 830 or other static storage device coupled to the I/O subsystem 820 for storing information and instructions for processor 810. The ROM 830 may include various forms of programmable ROM (PROM) such as erasable PROM (EPROM) or electrically erasable PROM (EEPROM). A unit of persistent storage 815 may include various forms of non-volatile RAM (NVRAM), such as FLASH memory, or solid-state storage, magnetic disk, or optical disk such as CD-ROM or DVD-ROM and may be coupled to I/O subsystem 820 for storing information and instructions. Storage 815 is an example of a non-transitory computer-readable medium that may be used to store instructions and data which when executed by the processor 810 cause performing computer-implemented methods to execute the techniques herein.

The instructions in memory 825, ROM 930 or storage 815 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 805 may be coupled via I/O subsystem 820 to at least one output device 835. In one embodiment, output device 835 is a digital computer display or Human Machine Interface. Examples of a display that may be used in various embodiments include a touch screen display or a light-emitting diode (LED) display or a liquid crystal display (LCD) or an e-paper display. Computer system 805 may include other type(s) of output devices 835, alternatively or in addition to a display device. Examples of other output devices 835 include printers, ticket printers, plotters, projectors, sound cards or video cards, speakers, buzzers or piezoelectric devices or other audible devices, lamps or LED or LCD indicators, haptic devices, actuators, or servos.

At least one input device 840 is coupled to I/O subsystem 820 for communicating signals, data, command selections or gestures to processor 810. Examples of input devices 840 include touch screens, microphones, still and video digital cameras, alphanumeric and other keys, keypads, keyboards, graphics tablets, image scanners, joysticks, clocks, switches, buttons, dials, slides.

Another type of input device is a control device 845, which may perform cursor control or other automated control functions such as navigation in a graphical interface on a display screen, alternatively or in addition to input functions. Control device 845 may be a touchpad, a mouse, a trackball, or cursor direction keys for communicating direction information and command selections to processor 810 and for controlling cursor movement on display 835. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 840 may include a combination of multiple different input devices, such as a video camera and a depth sensor.

In another embodiment, computer system 805 may comprise an internet of things (IoT) device in which one or more of the output device 835, input device 840, and control device 845 are omitted. Or, in such an embodiment, the input device 840 may comprise one or more cameras, motion detectors, thermometers, microphones, seismic detectors, other sensors or detectors, measurement devices or encoders and the output device 835 may comprise a special-purpose display such as a single-line LED or LCD display, one or more indicators, a display panel, a meter, a valve, a solenoid, an actuator or a servo.

Computer system 805 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 805 in response to processor 810 executing at least one sequence of at least one instruction contained in main memory 825. Such instructions may be read into main memory 825 from another storage medium, such as storage 815. Execution of the sequences of instructions contained in main memory 825 causes processor 810 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 815. Volatile media includes dynamic memory, such as memory 825. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 820. Transmission media can also take the form of acoustic or light waves, such as those generated during radio-wave and infra-red data communications.

Various forms of media may be involved in carrying at least one sequence of at least one instruction to processor 810 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 805 can receive the data on the communication link and convert the data to a format that can be read by computer system 805. For instance, a receiver such as a radio frequency antenna or an infrared detector can receive the data carried in a wireless or optical signal and appropriate circuitry can provide the data to I/O subsystem 820 such as place the data on a bus. I/O subsystem 920 carries the data to memory 825, from which processor 810 retrieves and executes the instructions. The instructions received by memory 825 may optionally be stored on storage 815 either before or after execution by processor 810.

Computer system 805 also includes a communication interface 860 coupled to bus 820. Communication interface 860 provides a two-way data communication coupling to network link(s) 865 that are directly or indirectly connected to at least one communication network, such as a network 870 or a public or private cloud on the Internet. For example, communication interface 860 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 870 broadly represents a local area network (LAN), wide-area network (WAN), campus network, internetwork, or any combination thereof. Communication interface 860 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 860 sends and receives electrical, electromagnetic, or optical signals over signal paths that carry digital data streams representing various types of information.

Network link 865 typically provides electrical, electromagnetic, or optical data communication directly or through at least one network to other data devices, using, for example, satellite, cellular, Wi-Fi, or BLUETOOTH technology. For example, network link 865 may provide a connection through a network 870 to a host computer 850.

Furthermore, network link 865 may provide a connection through network 870 or to other computing devices via internetworking devices and/or computers that are operated by an Internet Service Provider (ISP) 875. ISP 875 provides data communication services through a world-wide packet data communication network represented as internet 880. A server computer 855 may be coupled to internet 880. Server 855 broadly represents any computer, data center, virtual machine, or virtual computing instance with or without a hypervisor, or computer executing a containerized program system such as DOCKER or KUBERNETES. Server 855 may represent an electronic digital service that is implemented using more than one computer or instance and that is accessed and used by transmitting web services requests, uniform resource locator (URL) strings with parameters in HTTP payloads, API calls, app services calls, or other service calls. Computer system 805 and server 855 may form elements of a distributed computing system that includes other computers, a processing cluster, server farm or other organization of computers that cooperate to perform tasks or execute applications or services. Server 855 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 855 may comprise a web application server that hosts a presentation layer, application layer and data storage layer such as a relational database system using structured query language (SQL) or no SQL, an object store, a graph database, a flat file system or other data storage.

Computer system 805 can send messages and receive data and instructions, including program code, through the network(s), network link 865 and communication interface 860. In the Internet example, a server 855 might transmit a requested code for an application program through Internet 880, ISP 875, local network 870 and communication interface 860. The received code may be executed by processor 810 as it is received, and/or stored in storage 815, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 810. While each processor 810 or core of the processor executes a single task at a time, computer system 805 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

### OBJECT OF THE INVENTION

The present invention is intended to remedy all or part of the disadvantages of the prior art.

To this effect, according to a first aspect, the present invention aims at an aquatic installation four-dimensional monitoring system, comprising:
a submersible and/or floating vehicle, comprising:
   a relative positioning coordinates acquisition means, configured to locate, in a three-dimensional space representative of the aquatic installation, the submersible vehicle and to provide the corresponding coordinates of the submersible and/or floating vehicle, and
   a water physical and/or chemical sensor, configured to provide a measure of a local physical and/or chemical state of the aquatic installation in the proximity of the submersible and/or floating vehicle, and
a timestamping means, configured to associate, with relative positioning coordinates acquired, a value representative of the time of acquisition, and
an aquatic installation local physical and/or chemical state information aggregation means, configured to associate timestamped coordinates to a local measured physical and/or chemical state.

Thanks to these provisions, a multidimensional representation of a state of the aquatic installation at a given time may be obtained. Such a representation may be processed to evaluate the performance of water treatment processes interacting with the aquatic installation and, preferably, to use this evaluation in a feedback loop governing the operation of said water treatment processes. Such provisions further allow the more accurate and more detailed measurement of in-installation variations in physical and/or chemical state as opposed to global or biased overviews of current systems.

In particular embodiments, the system object of the present invention comprises an aquatic installation parameter determination means, configured to determine a value representative of a parameter of the aquatic installation or of the aquatic installation of said water as a function of several aggregated aquatic installation local physical and/or chemical state information.

Such embodiments allow the representation of the physical and/or chemical state of the aquatic installation to be processed to evaluate the performance of water treatment processes interacting with the aquatic installation and, preferably, to use this evaluation in a feedback loop governing the operation of said water treatment processes.

In particular embodiments, the aquatic installation parameter determination means operates a trained machine learning model.

In particular embodiments, the aquatic installation physical parameter determination means comprises an aquatic installation physical and/or chemical state uniformity determination means, configured to determine a value representative of a physical and/or chemical state uniformity of the water in the aquatic installation as a function of several aggregated aquatic installation local physical and/or chemical state information.

Such embodiments allow for the determination of the physical and/or chemical uniformity of the water in the installation. Such uniformity is representative of the capacity to impact the physical and/or chemical state of the water in an installation for a water treatment process or device associated with the installation.

In particular embodiments, the aquatic installation parameter determination means comprises a physical and/or chemical diffusion uniformity determination means, configured to determine a value representative of a physical parameter and/or of density uniformity of a chemical compound in the aquatic installation as a function of several aggregated aquatic installation local physical and/or chemical state information.

Such embodiments allow for the determination of the spread of a chemical compound in the water in the installation. Such uniformity is representative of the capacity to impact the chemical state of the water in an installation for a water treatment process or device associated with the installation.

In particular embodiments, the aquatic installation parameter determination means comprises a physical and/or chemical compound flow determination means, configured to determine a value representative of the flow of a physical and/or of a chemical compound in the aquatic installation as a function of several aggregated aquatic installation local physical and/or chemical state information.

Such embodiments allow for the determination of the flows in the water in the installation. Such flows are representative of the capacity to impact the physical and/or chemical state of the water in an installation for a water treatment process or device associated with the installation.

In particular embodiments, the aquatic installation parameters determination means comprises a risk zone determination means, configured to determine a value representative of a risk relative to the local physical and/or chemical state in at least part of the aquatic installation as a function of several aggregated aquatic installation local chemical state information.

Such embodiments allow for the determination of areas of the aquatic installation that may be at risk in regard to a particular criterion. Such a risk is representative of the capacity to impact the physical and/or chemical state of the water in an installation for a water treatment process or device associated with the installation.

In particular embodiments, the system object of the present invention comprises a command emitter configured to emit a command representative of a target operational value for an actuator interacting with the physical and/or chemical state of the aquatic installation.

Such embodiments allow for the constitution of a feedback loop.

In particular embodiments, the system object of the present invention comprises a remote computing device, comprising at least one of:
the timestamping means,
the information aggregation means and/or
the physical and/or chemical parameter determination means.

Such embodiments allow for the centralization, in a single location, of computing capacity that can be shared for several systems object of the present invention.

In particular embodiments, the system object of the present invention comprises both a submersible vehicle and a floating vehicle.

Such embodiments significantly increase the performance of the system, by providing surface-level sensing and in-depth sensing of physical and/or chemical parameters. In particular embodiments, at least one water physical and/or chemical sensor is an image acquisition means configured to acquire an image in which a color is representative of a local physical and/or chemical state of the aquatic installation.

Such embodiments allow for the determination of the presence of dirt or bacteria in the installation, for example.

In particular embodiments, at least one water physical and/or chemical sensor may be but is not limited to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

In particular embodiments, the water physical/chemical sensor 110 and/or the external sensor 118 is configured to measure a flow intensity from an inlet 112 in the installation, the aquatic installation state determination means 120 being configured to determine a pump flow efficiency as a function of the flow intensity.

In particular embodiments, at least one water physical and/or chemical sensor corresponds to an aquatic total alkalinity measurement system, comprising:
a pH probe configured to measure pH at the boundary layer of a body of water,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

Such provisions allow for the accurate and near real-time measurement of a total alkalinity value of a body of water at an inexpensive cost and with ordinary equipment. However, the benefits of this invention result from the counter-intuitive discovery, by the inventors, which switching the pH probe on and off when the water is not flowing provides an accurate total alkalinity measurement whereas continuous pH measurement does not. This is because the successive activation/deactivation, or connection/disconnection of the pH probe results in a chemical reaction taking place in the vicinity of the pH probe. This chemical reaction results in a variation in pH measurement, said variation being dependent on the total alkalinity value of the water in the vicinity of the pH probe off, when the water is not flowing. Therefore, the present invention allows for the determination of the total alkalinity value of a body of water without using a total alkalinity measurement sensor. Such an indirect measurement significantly improves the capacity to measure total alkalinity in swimming pools and in any other water installation and management systems.

In particular embodiments, the system object of the present invention comprises at least a sensor and/or actuator configured to interact with an element of an installation sanitation circuit associated to the aquatic installation, said sensor and/or actuator being activated as a function of at least one aggregated aquatic installation local physical and/or chemical state information.

Such embodiments allow for the constitution of a feedback loop.

According to a second aspect, the present invention aims at an aquatic installation four-dimensional monitoring method, comprising:
a step of operating a submersible and/or floating vehicle to navigate in an aquatic installation,
a step of relative positioning coordinates acquisition to locate, in a three-dimensional space representative of the aquatic installation, the submersible vehicle and to provide the corresponding coordinates of the submersible and/or floating vehicle,
a step of physical and/or chemical sensing to provide series of at least one sensed value representative of a local physical/chemical parameter in the proximity of the submersible and/or floating vehicle,
a step of timestamping to associate, with relative positioning coordinates acquired, a value representative of the time of acquisition and
a step of aquatic installation local physical and/or chemical state information aggregation to associate timestamped coordinates to a local measured physical and/or chemical state.

1. Aquatic installation four-dimensional monitoring system, comprising:
a submersible and/or floating vehicle, comprising:
a relative positioning coordinates acquisition means, configured to locate, in a three-dimensional space representative of the aquatic installation, the submersible vehicle and to provide the corresponding coordinates of the submersible and/or floating vehicle,
at least one physical and/or chemical sensor, configured to provide series of at least one sensed value representative of a local physical/chemical parameter in the proximity of the submersible and/or floating vehicle,
a timestamping means, configured to associate, with relative positioning coordinates acquired, a value representative of the time of acquisition, and
an aquatic installation physical and/or chemical state information aggregation means, configured to associate timestamped coordinates to a local measured physical and or chemical state.

2. System according to claim 1, which comprises an aquatic installation physical parameter determination means, configured to determine a value representative of a parameter of the aquatic installation or of the water in said installation as a function of several aggregated aquatic installation local physical and/or chemical state information.

3. System according to claim 2, in which the aquatic installation physical parameter determination means operates a trained machine learning model.

4. System according to claim 2, in which the aquatic installation parameter determination means comprises an aquatic installation physical and/or chemical state uniformity determination means, configured to determine a value representative of a physical and/or chemical state uniformity of the water in the aquatic installation as a function of several aggregated aquatic installation local physical and/or chemical state information.

5. System according to claim 2, in which the aquatic installation parameter determination means comprises a physical and/or chemical diffusion uniformity determination means, configured to determine a value representative of density uniformity of a physical parameter and/or of a chemical compound in the aquatic installation as a function of several aggregated aquatic installation local physical and/or chemical state information.

6. System according to claim 2, in which the aquatic installation parameter determination means comprises a physical parameter and/or a chemical compound flow determination means, configured to determine a value representative of the flow of a physical and/or of a chemical compound in the aquatic installation as a function of several aggregated aquatic installation local chemical state information.

7. System according to claim 2, in which the aquatic installation parameter determination means comprises a risk zone determination means, configured to determine a value representative of a risk relative to the local physical and/or chemical state in at least part of the aquatic installation as a function of several aggregated aquatic installation local physical and/or chemical state information.

8. System according to claim 2, which comprises a command emitter configured to emit an activation/deactivation command for an actuator interacting with the physical and/or chemical state of the aquatic installation to reach a target value for at least one physical and/or chemical parameter sensed.

9. System according to claim 1, which comprises a remote computing device, comprising at least one of:
the timestamping means, and/or
the information aggregation means, and/or
the physical and or chemical parameter determination means.

10. System according to claim 1, which comprises both a submersible vehicle and a floating vehicle.

11. System according to claim 1, in which at least one water physical and/or chemical sensor is an image acquisition means configured to acquire an image in which a color is representative of a local physical and/or chemical state of the aquatic installation.

12. System according to claim 1, in which at least one water physical and/or chemical sensor:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

13. System according to claim 1, in which at least one water physical and/or chemical sensor corresponds to an aquatic total alkalinity measurement system, comprising:
a pH probe configured to measure pH at the boundary layer of a body of water,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

14. System according to claim 1, which comprises at least a sensor and/or actuator configured to interact with an element of an installation sanitation circuit associated to the aquatic installation, said sensor and/or actuator being activated as a function of at least one aggregated aquatic installation local physical and/or chemical state information.

15. Aquatic installation four-dimensional monitoring method, comprising:
a step of operating a submersible and/or floating vehicle to navigate in an aquatic installation,
a step of relative positioning coordinates acquisition to locate, in a three-dimensional space representative of the aquatic installation, the submersible vehicle and to provide the corresponding coordinates of the submersible and/or floating vehicle,
a step of physical and/or chemical sensing to provide series of at least one sensed value representative of a local physical/chemical parameter in the proximity of the submersible and/or floating vehicle,
a step of timestamping to associate, with relative positioning coordinates acquired, a value representative of the time of acquisition, and
a step of aquatic installation local physical and/or chemical state information aggregation to associate timestamped coordinates to a local measured physical and/or chemical state.
